(19) United States
(12) Patent Application Publication
(10) Pub. No.: US 2005/0042179 A1
(43) Pub. Date: Feb. 24, 2005

(54) Inhalative powder formulations containing the CGRP-antagonist 1-[N2-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine

(75) Inventor: Michael Trunk, Ingelheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/909,619

(22) Filed: Aug. 2, 2004

Related U.S. Application Data
(60) Provisional application No. 60/503,012, filed on Sep. 15, 2003.

(30) Foreign Application Priority Data
Aug. 18, 2003 (DE) 103 38 407

(51) Int. Cl. A61K 38/04; A61K 31/517; A61L 9/04; A61K 9/14

(52) U.S. Cl. 424/46; 514/19; 514/252.17

(57) ABSTRACT

Powdered preparations for pulmonary or nasal inhalation, containing the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) or a pharmaceutically acceptable salt thereof, processes for preparing them and the use thereof for preparing a pharmaceutical composition for the treatment of headaches, migraine and cluster headache.

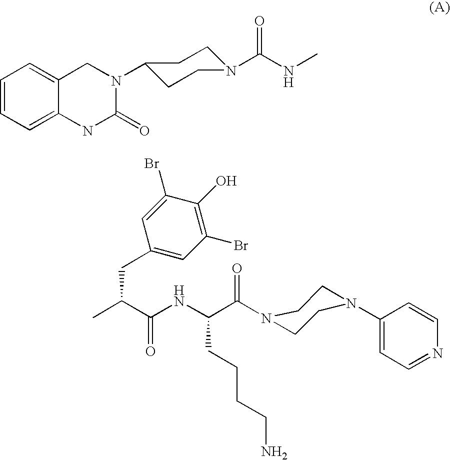

### RELATED APPLICATION

This application claims benefit of U.S. Ser. No. 60/503,012, filed Sep. 15, 2003, which is hereby incorporated by reference in its entirety.

### FIELD OF THE INVENTION

The invention relates to powdered preparations for pulmonary or nasal inhalation, containing the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) or a pharmaceutically acceptable salt thereof, processes for preparing them and the use thereof for preparing a pharmaceutical composition for the treatment of headaches, migraine and cluster headache.

### BACKGROUND TO THE INVENTION

The CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) is known from International Patent Application PCT/EP97/04862 (published as WO 98/11128) and has the following structure:

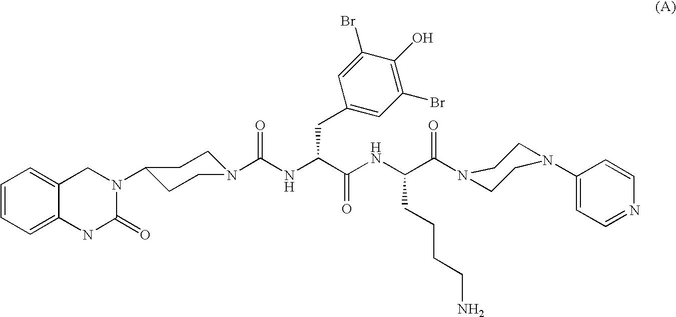

### PRIOR ART

The active substance base (A) is a highly effective CGRP antagonist for the acute and prophylactic treatment of headaches, particularly migraine and cluster headache, which cannot be administered orally using conventional formulations, as the substance has very limited oral bioavailability.

For treating sudden attacks of migraine it is essential that an active substance is systemically available as quickly as possible. The treatment should be uncomplicated for the patient to administer and no other conditions which could affect bioavailability (e.g. the food effect) should restrict the use of the medicament for the patient.

Active substances which are intended to be systemically available are usually administered by oral route. If this route is unsuitable or undesirable on account of particular properties of the active substance or particular demands made of the application, other possible ways of administering substances systemically are known in the art. For example, inhalation, by means of which active substances may be administered systemically as well as topically, has been under discussion for some time. For substances which prove critical on account of their decomposition in solution or which have poor solubility per se, powder inhalation is an option. The absolute amount of the active substance which has to be administered per application makes particular demands of the formulation. On the other hand, the physical stability (e.g. aerodynamic particle size, dispersibility, physicochemical properties) of the active substance has proved to be a critical requirement for the development and production of an inhalable powder.

With formulations of the powder inhalant type, inhalable powders, which are packaged for example in suitable capsules (inhalettes), are delivered to the lungs by means of powder inhalers. Similarly, other systems in which the quantity of powder to be administered is pre-dosed (e.g. blisters) and multidose powder systems are also known. Alternatively, the medicament may also be inhaled by the use of suitable powdered inhalable aerosols which are suspended for example in HFA134a, HFA227 or mixtures thereof as propellant gas.

In powder inhalation, the microparticles of a pure active substance are administered through the airways onto the surface of the lungs, e.g. in the alveoli, by the inhalation process. These particles settle on the surface and can only be absorbed into the body after the dissolving process by active and passive transporting processes.

Inhalation systems are known in the literature in which the active substance is present in the form of solid particles either as a micronised suspension in a suitable solvent system as carrier or in the form of a dry powder.

Usually, powder inhalants, e.g. in the form of capsules for inhalation, are prepared on the basis of the general teaching as described in DE-A-179 22 07. A critical factor in multi-substance systems of this kind is the uniform distribution of the pharmaceutical composition in the powder mixture.

Another important aspect of powder inhalants is that when the active substance is administered by inhalation only particles of a certain aerodynamic size reach the target organ, the lungs. The average size of these lung-bound particles (inhalable fraction) is in the region of a few microns, typically between 0.1 und 10 μm, preferably less than 6 μm. Such particles are usually produced by micronisation (air-jet grinding).

A formulation is only suitable for use as an inhalable powder containing the active substance if a micronised preparation and an excipient (carrier material) with special properties is used, the ratio of active substance to excipient is within a defined range and a specified amount of powder is available for the application. In addition, special climatic conditions must be adhered to during the manufacture of the pharmaceutical composition.

In the light of these fundamental technical requirements the problem is to propose solutions in accordance with the special properties of the active substance base 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) and the physiologically acceptable salts thereof by means of which the substances may be made sufficiently systemically bioavailable in the form of a powder inhalant.

### BRIEF DESCRIPTION OF THE INVENTION

The invention consists in providing a novel, stable formulation for the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine or a physiologically acceptable salt thereof, by means of which it is possible to produce adequate systemic blood levels for these substances which are not orally bioavailable. Similarly, the invention also includes methods of preparing such formulations and the use thereof for preparing a pharmaceutical composition.

It has been found that the active substance base 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) as well as the physiologically acceptable salts thereof are physically stable in the form of powder mixtures with excipients and may be made sufficiently bioavailable by pulmonary or nasal inhalation.

### DETAILED DESCRIPTION OF THE INVENTION

Surprisingly it has been found that the micronised active substance 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A), present in its amorphous form, or a physiologically acceptable salt thereof has proved to be physically stable in formulations of powder mixtures with a physiologically acceptable, homogeneous excipient. The powdered preparations described here enable the powder to be dispersed during inhalation and the active substance is made available for systemic administration by being taken in this way.

It is known that the amorphous state of solids is thermodynamically unstable. In particular, this contributes to the fact that microparticles which have amorphous parts or are purely amorphous are metastable in their physicochemical properties. Typically, amorphous or partially amorphous pharmaceutical active substances and excipients, such as sugars, for example, recrystallise spontaneously during storage under normal conditions. The process may be accelerated by an increase in the relative humidity and possibly the temperature. In association with this recrystallisation, the particles change their surface characteristics, their particle morphology and their particle size.

Surprisingly it has been found that the amorphous active substance 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) in the form of the amorphous micronised particles prepared therefrom (detected by X-ray crystallography/X-ray powder diffractometry) may be used to prepare a stable inhalable powder. The amorphous form of the active substance is maintained throughout the shelf life of the medicament.

According to the invention in addition to the active substance base the acid addition salts are used which are selected for example from among 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride, sulphate, phosphate, hydrobromide, carbonate, methanesulphonate, p-toluenesulphonate, nitrate, citrate, malate, tartrate, lactate, succinate, gluconate, acetate, formate, propionate, capronate, oxalate, maleate, fumarate, mandelate and hydroxysuccinate, while the 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride, the sulphate and the hydrobromide are particularly preferred and the 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride is most particularly preferred.

The formulations described here are designed so that the micronised active substance 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) with a particle size X₅₀ in the range from 1 μm to 6 μm, preferably from 1 μm to 3.5 μm, and a proportion of particles (by volume) Q_{(5.8)}<5.8 μm of at least 60%, is mixed with an inert excipient in a ratio of 1:9 to 5:1, in order to ensure a high active substance content according to the invention.

By the median value X₅₀ is meant the particle size below which 50% of the quantity of particles fall. The Q_{(5.8)} value describes the percentage of particles which are less than 5.8 μm in size.

It has been found that in order to prepare doses in which the dispersibility of the inhalable powder is sufficiently guaranteed for each inhalation, the micronised active substance may be combined with a coarser excipient (e.g. lactose) in the above proportions.

Powdered preparations of this kind are administered in amounts of from 25 mg to 100 mg, preferably 50 mg, per application by inhalation. During use it is also possible to achieve therapeutic blood levels by multiple administration.

Thus, in a first aspect the present invention relates to an inhalable powder for administration by pulmonary or nasal inhalation, containing as active substance the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) in the form of the active substance base or a physiologically acceptable salt thereof and an inert, homogeneous excipient, characterised in that
(a) the parameter X₅₀ for the particle size of the active substance in the range from 1 μm to 6 μm, preferably from 1 μm to 3.5 μm, and
(b) the characteristic value Q_{(5.8)} of the active substance is at least 60%.

Normal carrier materials or flow adjuvants may be used as physiologically acceptable homogeneous excipients according to the invention. The normal carrier materials may be selected from among the monosaccharides (e.g. glucose or arabinose), disaccharides (e.g. lactose, saccharose, maltose, trehalose), oligo- and polysaccharides (e.g. dextrans, starch, cellulose derivatives), polyalcohols (e.g. mannitol, sorbitol, xylitol), salts (e.g. sodium chloride, calcium carbonate), polylactides, polyglycolides and mixtures of these excipients. The flow adjuvants may for example be selected from a group consisting of magnesium stearate, calcium stearate, stearic acid, stearyl alcohols, calcium behenate, calcium arachinate, hydrogenated vegetable oils such as for example hydrogenated castor oil or hydrogenated cottonseed oil, fatty acid esters, sodium stearyl fumarate, sodium dodecyl sulphate, magnesium dodecyl sulphate and mixtures of these flow adjuvants.

The inhalable powders according to the invention may for example be administered using inhalers which meter a single dose from a reservoir by means of a measuring chamber (e.g. according to U.S. Pat. No. 4,570,630A) or using other equipment (e.g. according to DE 36 25 685 A). Preferably, however, the inhalable powders according to the invention are packed into capsules (to form so-called inhalettes), which are used in inhalers as described, for example, in WO 94/28958.

The inhalable powders according to the invention may be obtained by the method described below.

As the active substance base (A) as well as the salts thereof are hygroscopic, particular ambient conditions must be maintained when weighing out these substances.

After suitable micronisation of the active substance it is conditioned at a specified temperature and humidity and in this way an equilibrium is achieved between the water content of the active substance and the relative humidity of the environment. Then the conditioned active substance is suitably mixed with one or more excipients and the amount of the resulting powder mixture which is to be administered is packaged in single doses under defined climatic conditions (temperature and humidity) taking into account the water content of the active substance obtained according to these conditions (corrected weight). The mixture is packed into inhalettes which are later used in inhalers suitable for this purpose. The preparation of the inhalable powders is then followed by the production of the powder-filled capsules, which have to be put into their final packaging (blister backs) in suitable manner.

In a second aspect the present invention relates to a process for preparing a powder inhalant according to the invention, characterised in that
(a) the active substance is micronised,
(b) the micronised active substance is conditioned,
(c) suitably mixed with one or more excipients according to the invention and
(d) the amount of the resulting powder mixture which is to be administered is packaged as single doses in inhalettes under defined climatic conditions.

The powder mixture according to the invention may be inhaled, the powder preferably being presented to the patient in the form of a pre-metered pharmaceutical preparation. One example of this is an inhalable capsule system. Also possible are systems in which the powder preparation is provided, for example, in the form of single doses backed into wells in blister packs. The powdered preparations described here may be inhaled and thus delivered to the lungs using a suitable device.

The inhalable powder containing the active substance which may be produced from such preparations has a particle size which is characterised in that the active substance base 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) or the physiologically acceptable salts thereof is or are in the form of microparticles of a size suitable for penetration into the lungs. At the same time, it is found that powder mixtures such as may be obtained according to the description of this formulation are sufficiently easy to process, in terms of their cohesive properties, to reproducibly form a pharmaceutical composition. It is thus possible to design a powdered preparation for use in pulmonary (and optionally nasal) inhalation such that on the one hand, during the dispersion of the powder in the course of the inhalation process by the patient, the amorphous active substance has an aerodynamic particle size which leads to settling of the active substance in the lungs after nasal or pulmonary inhalation, and on the other hand the powder (consisting of the micronised active substance and a carrier material) is designed to be suitable for processing by machine. Thanks to this administration of the active substance by nasal or pulmonary inhalation in the lungs, which can be achieved using this technique, the active substance has sufficient systemic bioavailability.

It has also been found that, surprisingly, a micronised preparation of the active substance base 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A), which may be prepared for example by known methods such as air-jet grinding, is only suitable if in addition to the above-mentioned conditions relating to particle size it also has special properties with regard to the specific surface area of (A) in relation to the surface area of the inert excipient of the formulation. It is found that the formulation is particularly suitable if the quotient of the specific surface area of the micronised preparation of (A) to the specific surface area of the inert excipient, based in each case on the total quantity of powder available per application, is greater than 0.05, preferably greater than 0. 1, particularly preferably greater than 0.5, most particularly preferably greater than 0.7, and in each case is less than 22, preferably less than 15.

In addition to the use of air-jet-ground 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) it is also suitable to use a micronised preparation of (A) which is present in a particle size as stated above, produced by alternative methods. Therefore, such formulations may also be prepared for example using micronised preparations of the active substance (A) or a physiologically acceptable salt thereof, which are prepared by spray-drying, irrespective of whether this spray micronised preparation is obtained as a single-component system or in the form of spray particles consisting of the active substance and one or more excipients.

Powdered preparations which consist of components that satisfy the above requirements regarding the particle size of the active substance and the ratio of the specific surface area of the active substance to the excipient can be processed by known methods to form homogeneous powder mixtures and packed by known methods into capsules or other systems for pre-dosing. However, such manufacturing steps are only successful when the handling of the powder takes place under strict climate control. For the successful production the maximum temperature difference and the band width within which the relative humidity should fluctuate during the particular manufacturing step are critical as the active substance according to the invention is strongly hygroscopic. Ideally, the temperature should differ by not more than ±5° C., preferably ±3° C., either side of a freely selected mean value and the humidity should fluctuate by not more than ±15%, preferably ±10%, either side of the freely selected mean value. Adjusting the amount of active substance weighed out (weight correction) should be carried out after adjusting the equilibrium between the relative humidity of the environment and the water content of the active substance, depending on the hygroscopicity of the active substance.

In a third aspect the present invention relates to the use of an inhalable powder according to the invention as a pharmaceutical composition, particularly for preparing a pharmaceutical composition for the treatment of headaches, migraine or cluster headache.

In a fourth aspect the present invention relates to the use of an inhalable powder according to the invention for preparing a capsule (inhalette). Such a capsule (inhalette) is characterised by a content of 2 to 50 mg of inhalable powder according to the invention.

### Experimental Section

### 1) Methods of Measurement

a) Determining the Particle Size by Laser Diffraction (Frauenhofer Diffraction):

Measuring method: In order to determine the particle size the powder is fed into a laser diffraction spectrometer using a dispersing unit. The median value X₅₀ refers to the particle size below which 50% of the quantity of particles fall. The Q_{(5.8)} value describes the percentage of particles which are less than 5.8 μm in size.

Measuring device: Laser diffraction spectrometer (HELOS), Messrs. Sympatec

Software: WINDOX Version 3.3/REL 1

Dispersing unit: RODOS/dispersing pressure: 3 bar

Focal length: 100 mm [measuring range: 0.9 . . . 175 μm]

Evaluation method: HRLD (V 3.3 Rel. 1)

b) Determining the Specific Surface Area:

Measuring method: The specific surface is determined by exposing the powder sample to a nitrogen atmosphere at different pressures. Cooling the sample causes the nitrogen molecules to be condensed on the surface of the particles. The quantity of condensed nitrogen is determined by means of the drop in pressure in the system and the specific surface area of the sample is calculated by means of the surface nitrogen requirement and the weight of the sample.

Measuring device: Tri Star Multi Point BET, Messrs. Micromeritics

Heating station: VacPrep 061, Messrs. Micromeritics

Heating: approx. 12 h/40° C.

Analysis parameters

| | |
|---|---|
| sample tube: | ½ inch; with filler rod |
| analysis method: | 10 point BET surface measurement |
| | 0.1 to 0.20 p/p0 |
| absolute pressure tolerance: | 5.0 mm Hg |
| relative pressure tolerance: | 5.0% |
| evacuation rate: | 50.0 mm Hg/second |
| evacuation threshold: | 10.0 mm Hg |
| evacuation time: | 0.1 h |
| free space: | lower Dewar, t: 0.5 h |
| retention time: | 20 seconds |
| minimum equilibration delay: | 600 seconds |
| adsorptive: | nitrogen |

### EXAMPLES

a) 50 g of (anhydrous) air-jet-ground active substance with a specific surface area of 20.2 m²/g are conditioned at 25° C. and 45% relative humidity for 8 hours and mixed with 450 g Pharmatose® 200M (manufactured by Danone), specific surface area 0.96 m²/g (screened in layer by layer, Turbula mixer).

Under the same ambient conditions as for the preparation of the starting materials and the mixing of the individual components the mixture is transferred into single capsules. A filling of 20.12 mg of the powder mixture with the above composition corresponds to a micronised active substance content per capsule of 2 mg (anhydrous).

b) 100 g (anhydrous) air-jet-ground active substance with a specific surface area of 20.2 m²/g are conditioned at 25° C. and 45% relative humidity for 8 hours and mixed with 200 g Pharmatose® 325M (manufactured by DMV), specific surface area 0.25 m²/g (screened in layer by layer, Turbula mixer).

Under the same ambient conditions as for the preparation of the starting materials and the mixing of the individual components the mixture is transferred into single capsules. A filling of 48.96 mg of the powder mixture with the above composition corresponds to a micronised active substance content per capsule of 16 mg (anhydrous).

c) 200 g (anhydrous) spray-dried active substance with a specific surface area of 7.8 m²/g are conditioned at 25° C. and 30% relative humidity for 8 hours and mixed with 200 g Lactochem® Super Fine Powder (manufactured by Borculo), specific surface area 0.75 m²/g (screened in layer by layer, Turbula mixer).

Under the same ambient conditions as for the preparation of the starting materials and the mixing of the individual components the mixture is transferred into single capsules. A filling of 51 mg of the powder mixture with the above composition corresponds to a micronised active substance content per capsule of 25 mg (anhydrous).

d) 400 g (anhydrous) spray-dried active substance with a specific surface area of 1.4 m²/g are conditioned at 25° C. and 30% relative humidity for 8 hours and mixed with 100 g Pharmatose® 200M (manufactured by Danone), specific surface area 0.96 m²/g (screened in layer by layer, Turbula mixer).

Under the same ambient conditions as for the preparation of the starting materials and the mixing of the individual components the mixture is transferred into single capsules. A filling of 51.6 mg of the powder mixture with the above composition corresponds to a micronised active substance content per capsule of 40 mg (anhydrous).

1. An inhalable powder for administration by pulmonary or nasal inhalation, containing as active substance the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine 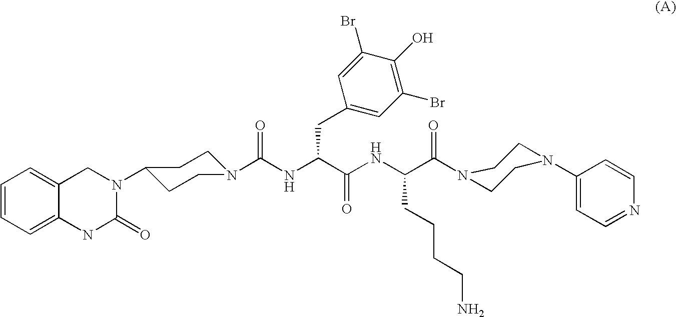
or a physiologically acceptable salt thereof and an inert, homogeneous excipient, characterised in that
(a) the parameter X₅₀ for the particle size of the active substance is in the range from 1 μm to 6 μm, preferably from 1 μm to 3.5 μm, and
(b) the characteristic value Q_{(5.8)} for the active substance is at least 60%.

2. The inhalable powder according to claim 1, characterised in that the active substance base 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) is present in the form of an amorphous micronised preparation.

3. The inhalable powder according to claim 1, characterised in that the physiologically acceptable salt is selected from the group consisting of 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride, sulphate, phosphate, hydrobromide, carbonate, methanesulphonate, p-toluenesulphonate, nitrate, citrate, malate, tartrate, lactate, succinate, gluconate, acetate, formate, propionate, capronate, oxalate, maleate, fumarate, mandelate and hydroxysuccinate.

4. The inhalable powder according to claim 1, characterised in that the physiologically acceptable salt is selected from the group consisting of 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride, sulphate and hydrobromide.

5. The inhalable powder according to claim 1, characterised in that the physiologically acceptable salt is the 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride and is present as an amorphous micronised preparation.

6. The inhalable powder according to claim 1, characterised in that the excipients used are monosaccharides, disaccharides, oligo- and polysaccharides, polyalcohols, salts, polylactides, polyglycolides or mixtures of these excipients with one another.

7. The inhalable powder according to claim 6, characterised in that the excipients used are glucose, arabinose, lactose or saccharose, maltose, trehalose, dextrans, starch, cellulose derivatives, mannitol, sorbitol, xylitol, sodium chloride, calcium carbonate, polylactides, polyglycolides or mixtures of these excipients with one another.

8. The inhalable powder according to claim 1, characterised in that flow adjuvants are used as excipients.

9. The inhalable powder according to claim 8, characterised in that the flow adjuvants used are magnesium stearate, calcium stearate, stearic acid, stearylalcohols, calcium behenate, calcium arachinate, hydrogenated vegetable oils, fatty acid esters, sodium stearyl fumarate, sodium dodecyl sulphate, magnesium dodecyl sulphate or mixtures of these flow adjuvants.

10. The inhalable powder according to claim 1, characterised in that the ratio of active substance to excipient is from 1:9 to 5:1.

11. The inhalable powder according to claim 1, characterised in that the quotient of the specific surface area of the active substance to the specific surface area of the inert excipient is greater than 0.05, preferably greater than 0.1, particularly preferably greater than 0.5, most particularly preferably greater than 0.7, and less than 22, preferably less than 15, based in each case on the total quantity of powder available per application.

12. A process for preparing an inhalable powder for administration by pulmonary or nasal inhalation, containing as active substance the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine 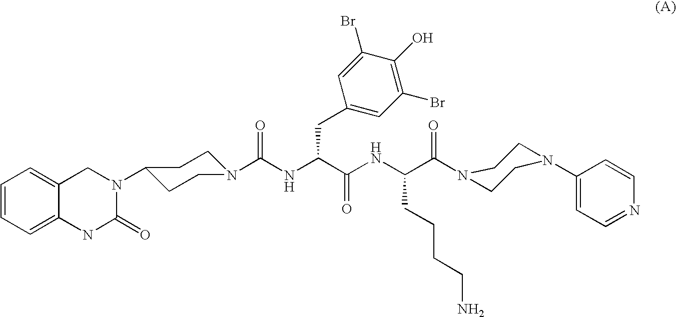
or a physiologically acceptable salt thereof and an inert, homogeneous excipient, characterised in that
(b) the active substance is micronised,
(b) the micronised active substance is conditioned,
(c) suitably mixed with one or more excipients according to the invention and
(d) the amount of the powder mixture thus obtained which is to be administered is packaged in inhalettes in single doses under specified ambient climatic conditions.

13. The product of the process of claim 12.

14. A method for treating headache, migraine or cluster headache which comprises administering the inhalable powder of claim 1.

15. A capsule (inhalette) containing the inhalable powder of claim 1.

16. The capsule (inhalette) of claim 15, characterised in that it contains a filling of 2 mg to 50 mg of an inhalable powder.

17. The capsule (inhalette) according to claim 16, characterised in that it contains between 2 mg and 50 mg of 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A).
